(19) United States
(12) Patent Application Publication
(10) Pub. No.: US 2020/0155647 A1
(43) Pub. Date: May 21, 2020

(54) NEUROTROPHINS FOR USE IN THE TREATMENT OF HEARING LOSS

(71) Applicant: DOMPÉ FARMACEUTICI S.P.A., MILANO (IT)

(72) Inventors: Maria DE PIZZOL, Milano (IT); Andrea ARAMINI, L'Aquila (IT); Anna SIRICO, Somma Vesuviana (IT); Mara ZIPPOLI, Genova (IT); Giuseppina ACERRA, Lauro (IT); Simone Luca MATTIOLI, Milano (IT)

(21) Appl. No.: 16/615,201

(22) PCT Filed: May 22, 2018
(86) PCT No.: PCT/EP2018/063310

§ 371 Date: Nov. 20, 2019

(30) Foreign Application Priority Data
May 24, 2017 (EP) 17172659.9

(51) Int. Cl. A61K 38/18; A61K 9/00; A61K 9/16; A61K 31/573

(52) U.S. Cl. CPC A61K 38/185; A61K 31/573; A61K 9/1647; A61K 9/0046

(57) ABSTRACT

The present invention relates to the use of neurotrophins in the treatment of hearing disorders related to hearing loss using the transtympanic route of administration, in particular in the treatment of sudden deafness, blast-induced hearing loss, ototoxicity, ARHL (age related hearing loss) and noise damage. The invention further relates to the use of compositions containing neurotrophins, in particular NGF, in the treatment of hearing disorders.

The present invention relates to the use of neurotrophins in the treatment of hearing disorders related to hearing loss using the transtympanic route of administration, in particular in the treatment of sudden deafness, blast-induced hearing loss, ototoxicity, ARHL (age related hearing loss) and noise damage. The invention further relates to the use of compositions containing neurotrophins, in particular NGF, in the treatment of hearing disorders.

### BACKGROUND OF THE INVENTION

Deafness occurs in 0.1-0.2% of newborns, and hearing loss affects up to 70% of the population over the age of 75 years (Sprinzl G M et al., Gerontology 2010; 56(3):351-8). Sound signals are converted into electrical impulses by auditory hair cells located in the cochlea. Degeneration of the hair cells, due to infections, mechanical injury, aminoglycoside antibiotics, acoustic trauma, aging and chemical-induced ototoxicity, is a major cause of hearing impairments or loss (Delterne P et al., Acta Otolaryngol 1993; 113:312-377).

Ototoxic drugs include the commonly used aminoglycoside antibiotics, such as gentamicin, for the treatment of infections caused by Gram-negative bacteria, the widely used chemotherapeutic agent cisplatin and its analogues, quinine and its analogues, salycilate and its analogues, and loop-diuretics. The toxic effects of these drugs on auditory cells and spiral ganglion neurons are often the limiting factor for their therapeutic usefulness. For example, antibacterial aminoglycosides such as gentamicin and the like are known to have serious toxicity, particularly ototoxicity and nephrotoxicity, which reduce the usefulness of such antimicrobial agents (Goodman and Gilman's The Pharmacological Basis of Therapeutics, Vol 70, Issue 5, May 1981, page 581-6^{th} ed.).

Otitis media is a term used to describe infections of the middle ear. Typically antibiotics are systemically administered for infections of the middle ear. Systemic administration of antibiotics generally results in a prolonged lag time to achieve therapeutic levels in the middle ear and requires high initial doses in order to achieve such levels. Systemic administration is most often effective when infection has reached advanced stages, but at this point permanent damage may already have been caused to the middle and inner ear structure. Clearly, ototoxicity is a dose-limiting side-effect of antibiotic administration.

Auditory apparatus can be divided into the external and middle ear, inner ear and auditory nerve and central auditory pathways. While having some variations from species to species, the general characterization is common for all mammals. Auditory stimuli are mechanically transmitted through the external auditory canal, tympanic membrane, and ossicular chain to the inner ear.

Many studies have proposed various drugs and agents as potential therapeutic interventions in ototoxicity, ARHL (Age Related Hearing Loss) and noise damage. These broadly fall into two categories: those aimed at inhibiting specific parts of the cell death pathways, and those aimed at interrupting the signaling mechanism leading to apoptosis and necrosis. Several strategies of intervention are based on the reduction of ROS (Reactive Oxygen Species) by using anti-oxidant drugs such as N-acetylcysteine (NAC), already used in some clinical trials.

In the last years many studies concerning hair cell regeneration were conducted. Many of them have shown that supporting cells of the mammalian organ of Corti can be induced to division after specific gene manipulation. Recent evidence suggests that the postnatal mammalian inner ear contains progenitor cells, which are able to proliferate In vitro or form otospheres and differentiate into multiple phenotypes.

Rehabilitative measures traditionally used are based on technical solutions, primarily using hearing aids to amplify and filter incoming sounds signals, while in cases of more severe impairment an option is offered by the cochlear prosthesis, which directly stimulates the auditory neurons. Altering expression of specific genes responsible for the differentiation of hair cells (Mizutari K et al., Neuron 2013; 77(1):58-69) or stem cell therapy (Okano T et al., Trends Amplif. 2012; 16(1):4-18) is likely the solution for hearing function restoration. Methods or strategies for hair cell protection able to delay the degeneration process are also actively pursued. However an effective clinical treatment has not yet been found.

The NGF family of neurotrophins is a class of emerging molecules required for the inner ear innervation in mammals (Ernfors P et al, Neuron 1995:14:1153-64); their potential therapeutic application in humans is actively under evaluation. This family includes the nerve growth factor (NGF), the brain-derived neurotrophic factor (BDNF) and the neurotrophins 3 and 4 (NT3, NT4). These four molecules share a similar sequence and structure, as they all descend from a common ancestral gene, and all four are active in directing neurons growth and differentiation during development and beyond (Huang E J et al., Annu Rev Neurosci 2001; 24:677-736). Each neurotrophin binds to a specific subtype of Trk receptor with high affinity, and some low affinity cross binding to other Trk receptors and to p75 receptor also occurs.

The brain-derived neurotrophic factor (BDNF) acts on certain neurons of the central nervous system and the peripheral nervous system, helping to support the survival of existing neurons, and encourage the growth and differentiation of new neurons and synapses.

Neurotrophin 3 is a protein growth factor which has activity on certain neurons of the peripheral and central nervous system; it helps to support the survival and differentiation of existing neurons, and encourages the growth and differentiation of new neurons and synapses.

Neurotrophin 4 is a neurotrophic factor that signals predominantly through the TrkB receptor tyrosine kinase.

NGF is a polypeptide composed of three subunits, α, β and γ. The β-subunit itself is a homodimer of peptides composed by 118 amino acids and is responsible for the full biological activity of NGF. Its structure is well preserved among different species, with 90% homology between murine and human NGF. NSF regulates growth and survival of nerve cells, profoundly affecting the development of both young and adult nervous system.

In adults, NGF was shown to be effective in pain/hyperalgesia, as well as in several eye diseases, such as neurotrophic keratitis, optic nerve transection, ocular hypertension and retinal detachment. Said data have been reported mainly for NSF extracted from murine salivary glands, even these preparations are heterogeneous mixtures of different dimers.

Recently, human NSF was synthesized using genetic engineering techniques (Iwane et al., Biochem. Biophys. Res. Commun., 171: 116, 1990; EP099188B1, WO2013/092776), with the advantage to obtain a more homogeneous form of protein to be administered to patients. Various animal models were used to stimulate and investigate the morphological and physiologic effects of the insults to the inner ear. When administered to deafened cochlea in animal models, neurotrophins, showed to be able to dramatically improve spiral ganglion neuron survival and to stimulate peripheral auditory fiber regrowth.

Furthermore, patients affected by blast-induced hearing loss and treated with NGF by intramuscular injection showed a hearing improved when early treated (S.Q. Zhai, N. YU. Eur. Rev. Med. Pharm. Sci., 2015:19:3146-51). A therapeutic effect was observed following NGF-point injections in the treatment of nervous deafness and tinnitus (Shanxi et al., J. Trad Chinese Med 2009; 29:39-42).

Although the parenteral administration of NGF, in particular by intramuscular and intravascular route, is needed when a prolonged NGF action is wanted, said route of administration does not solve the main issue to reach high doses of NGF in the inner ear, in particular to achieve a therapeutic and efficacious intralabyrinth concentration. Furthermore, several known strategies for NGF delivery to the inner ear are known, that include systemic and intravenous administration, but these routes are not selective, and therapeutic concentrations are difficult to be achieved inside the inner ear.

There is therefore the need of a more specific and localized treatment able to reach the inner ear with a concentration of drug sufficiently high to guarantee the therapeutic efficacy of NGF.

### Definitions

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference; thus, the inclusion of such definitions herein should not be construed to represent a substantial difference over what is generally understood in the art.

The term “pharmaceutically acceptable excipient” herein refers to a substance devoid of any pharmacological effect of its own and which does not produce adverse reactions when administered to a mammal, preferably a human. Pharmaceutically acceptable excipients are well known in the art and are disclosed, for instance in the Handbook of Pharmaceutical Excipients, sixth edition 2009, herein incorporated by reference.

The term “simultaneous, separate or sequential administration” herein refers to administration of the first and second compound at the same time or in such a manner that the two compound act in the patient's body at the same time or administration of one compound after the other compound in such a manner to provide a therapeutic effect.

The terms “approximately” and “about” herein refers to the range of the experimental error, which may occur in a measurement.

The terms “comprising”, “having”, “including” and “containing” are to be construed open-ended terms (i.e. meaning “including, but not limited to”) and are to be considered as providing support also for terms as “consist essentially of”, “consisting essentially of”, “consist of” or “consisting of”.

The terms “consist essentially of”, “consisting essentially of” are to be construed as semi-closed terms, meaning that no other ingredients which materially affects the basic and novel characteristics of the invention are included (optional excipients may thus included).

The terms “consists of”, “consisting of” are to be construed as closed terms.

The term “intratympanic administration” means a surgical procedure in which a small amount of a drug is injected directly into the middle ear through the tympanic membrane. It is usually performed at the postero-inferior quadrant of the tympanic membrane which is at the level of the round window, “the gate to the inner ear”, because it hosts a membrane permeable to some drugs.

The term “controlled release” herein refers to the control of the rate and/or quantity of biologically active molecules delivered according to the drug delivery formulations of the invention. The controlled release kinetics can be prolonged release, fast release, delayed release or pulsatile drug delivery system.

### DESCRIPTION OF THE FIGURES

FIG. 1. Cell survival after rhNGF treatment of damaged cochlea.

FIG. 2, rhNGF—MPS in vitro cumulative release profile from the formulation 2.

FIG. 3. Amount of rhNGF (ng) released day by day from formulation 2.

### DESCRIPTION OF THE INVENTION

It was surprisingly found that the local neurotrophin delivery approach including the intratympanic administration, that limits systemic exposure, is particularly promising in the treatment of hearing loss, because it allows the neurotrophin to reach adequate therapeutic levels into the inner ear, particularly in the intra labyrinthique region, faster and at higher concentrations.

This route of administration provides for better results than other common routes, such as intravenous or oral administration. The obtained results are imputable to the neurotrophin activity in restoring hair cells viability after gentamicin treatment, and to the transtympanic administration leading to a good distribution of neurotrophin into the cochlea.

In particular, the experimental data reported in the present invention shows that the transtympanic administration of rhNGF is able to regenerate the inner ear tissue, particularly the inner ear epithelial hair cells, and therefore represents an improvement over the existing treatments and an alternative to corticosteroid treatment both by oral and parenteral administration.

Therefore, a first embodiment of the present invention relates to the use of a neurotrophin in the treatment of hearing disorders, characterized in that said neurotrophin is administered by intratympanic route.

In a preferred embodiment of the present invention said neurotrophin is selected from NGF, brain-derived neurotrophic factor (BDNF), neurotrophin 3 and neurotrophin 4, more preferably NGF.

Preferably, said hearing disorders are selected from sudden deafness, blast-induced hearing loss, ototoxicity, ARHL (age related hearing loss) and noise damage.

In a preferred embodiment, said neurotrophin is administered by means of a composition further comprising at least one pharmaceutically acceptable excipient.

Preferably, the administered neurotrophin is NGF, brain-derived neurotrophic factor (BDNF), neurotrophin 3 and neurotrophin 4, more preferably NGF.

For the purpose of the present invention pharmaceutically acceptable excipient suitable for intratympanic administration are selected from hyaluronic acid, polyvinyl alcohol (PVA), glycerol, poly-lactic-co-glycolic acid (PLGA) and PEG400, preferably hyaluronic acid, polyvinyl alcohol or glycerol.

In a preferred embodiment of the present invention, said composition is a solution, a suspension, microparticles or a gel. Said microparticles are preferably made of poly-lactic-co-glycolic acid (PLGA).

Preferably, when the composition is a gel, said gel is selected from a thereto-sensitive gel or an adhesive sal-gel transition hydrogels. More preferably said gels include Pluronic F127 and Hyaloronic acid (HA).

In a further preferred embodiment of the present invention said neurotrophin is administered by means of a controlled release composition comprising solid microparticles of PLGA, at least one organic solvent and at least one additional molecule, selected from human serum albumin (HAS), polyethylene glycol (PEG) and trehalose.

Preferably said organic solvent is non halogenated (e.g. fluorinated or chlorinated). More preferably said non halogenated organic solvent is selected from the group consisting of ethyl acetate and acetone.

Preferably said composition of microparticies contains albumine from human serum, ethyl acetate and D-trehalose dehydrate.

Preferably said controlled release composition is a slow release composition.

In a further embodiment the composition of the present invention can be administered simultaneously, separately or sequentially in combination with at least one further active principle, also following different route of administration for each active principle. In a preferred embodiment said at least one further active principle is a steroid, selected from dexamethasone, methylprednisolone and prednisolone.

Preferably, said further active principle can be administered orally, intramuscularly or topically.

In a further embodiment, the composition of the present invention contains from 1 μg to 1.2 mg of neurotrophin, preferably from 10 μg to 200 μg of neurotrophin, more preferably from 2 μg to 20 μg of neurotrophin.

Preferably said neurotrophin is NGF, brain-derived neurotrophic factor (BDNF), neurotrophin 3 and neurotrophin 4, more preferably NGF.

According to a preferred embodiment, the composition of the present invention contains from 1 μg to 1.2 mg of NGF, preferably from 10 μg to 200 g of NGF, more preferably from 2 μg to 20 μg of NGF.

When the composition of the present invention is in form of a suspension the concentration of neurotrophin is from 1 μg/ml to 1.2 mg/ml, preferably from 10 μg/ml to 200 μg/ml, more preferably from 2 μg/ml to 20 μg/ml of neurotrophin.

Preferably said neurotrophin is NSF, brain-derived neurotrophic factor (BDNF), neurotrophin 3 and neurotrophin 4, more preferably NSF.

In a more preferred embodiment in said suspension the concentration of NSF is from 1 μg/ml to 1.2 mg/ml, preferably from 10 to 200 μg/ml, more preferably from 2 μg/ml to 20 μg/ml of NSF.

When the composition of the present invention is in form of a solution the concentration of neurotrophin is from 1 μg/ml to 1.2 mg/ml, preferably from 10 μg/ml to 200 μg/ml, more preferably from 2 μg/ml to 20 μg/ml of neurotrophin.

Preferably said neurotrophin is NGF, brain-derived neurotrophic factor (BDNF), neurotrophin 3 and neurotrophin 4, more preferably NGF.

When the composition of the present invention is in form of a solution the concentration of NSF is from 1 μg/ml to 1.2 mg/ml, preferably from 10 μg/nil to 200 μg/ml, more preferably from 2 μg/ml to 20 μg/ml of NSF.

When the composition of the present invention is in form of microparticles the concentration of NGF is from 1 μg/ml to 1.2 mg/ml, preferably from 10 g/ml to 200 μg/ml, more preferably from 2 μg/ml to 20 μg/ml of NSF.

In a further embodiment the neurotrophin of the present invention is of murine or human origin or it is a human recombinant neurotrophin.

Preferably, said neurotrophin is a NSF of murine or human origin or is a human recombinant NSF.

According to a preferred embodiment, the composition for use of the present invention is administered daily, preferably one or more time a day.

Preferably, said composition for use is administered from one to four doses a day, for at least 4 weeks.

According to the invention, the composition of the present invention may be administered to humans, intended to comprise both adults and the “pediatric population” (where the term “pediatric population” is understood as the part of the population ranging from birth to eighteen years of age).

The following examples are included to increase the understanding of the invention, without having any limiting effect of the invention.

### Experimental Data

The following formulations according to the present invention were prepared.

### Formulation 1

### Microparticles Slow Release

The PLGA/NGF microparticles of formulation 1 have been prepared as follow. The yield is of 74.31% with a theoretical amount of 0.0088% (mg NGF/100 mg nip). A 100 μl aqueous solution containing NGF (50 μl), HSA (1.99 mg) and PEG400 (50 μl) (1:40:1 ratio, vol./weight/vol.) was emulsified for 1 minute in 2.5 ml of 20% solution of PLGA 752H in Ethyl Acetate containing 0.5 g of polymer by means of an Ultraturrax homogenizer T25 Basic (IKA), with 8G probe, set at 17500 rpm. The obtained emulsion (A1/O) was rapidly added to 10 ml of an aqueous solution PVA 1% p/v (Mowiol®, 40-88) mixed with 300 μl of ethyl acetate and homogenized at 12600 rpm for 30 minutes. The resulting double emulsion (A1/O/A2) was left under electromagnetic stirring for 3 hours at room temperature using an RW20D (IKA) rod stirrer, set at 500 rpm, to facilitate evaporation of the organic solvent and precipitation of PLGA particles. The suspension was collected in a 50 ml tube and centrifuged at 7000 rpm for 15 min at 4° C.

The isolated microparticles were washed 4 times with 50 ml of MilliQ water, centrifuged (7000 rpm, 4° C., 15 minute), and then resuspended in 3 ml of 2% w/v aqueous trealose solution. The obtained suspension was transferred into 2 double silicone glass vials, frozen at −80° C., then subjected to a 24 hour lyophilization cycle.

TABLE 1
Microparticles components - formulation 1

| Components | Quantity | |
|---|---|---|
| rhNGF | 0.0495 | mg |
| PLGA 752H | 2.5 | ml |
| PEG 400 | 0.05 | ml |
| HSA | 1.99 | mg |
| Ethyl acetate | 2.8 | ml |
| Polyvinyl alcohol 40-88 | 10 | ml |
| Trehalose | 3 | ml |

### Formulation 2

TABLE 2
Microparticles components - formulation 2

| | Amount | | | |
|---|---|---|---|---|
| PRODUCT | Volume (mL) | Weight (mg) | Density(g/cm³) | % |
| NGF | 0.15 | 0.129 | | |
| PLGA 752H sol. | 7.5 | 1500 | | 20 |
| PEG 400 | 0.15 | 0.1689 | 1.126 | |
| HSA | | 6.05 | | |
| Ethyl acetate | 8.4 | | | |
| Polyvinil Alchool | 30 | | | 1 |
| 40-88 | | | | |
| Trehalose | 9 | 180 | | 2 |

### Features of the Microparticles

Particle size distribution (PSD) 3 μm≤PSD(0.5)≤15 μm

Morphology smooth-surface spherical particles

Encapsulation efficiency >50% (about 65%)

Controlled release ≥50 gg (mean 20 ng/die of rhNGF)

Biological activity (potency) 70-130% (of reference material)

### Preparation Procedure of Formulation 2.

PLGA/rhNGF microparticles with theoretical amount of 0.0076% (mg rhNGF/100 mg mps) are prepared in this way: 300 μl aqueous solution, containing rhNGF solution in formulation buffer (FB) (150 μl), HSA (6.01 mg), and PEG400 (150 μl) (ratio 1:40:1, v/w/v) was emulsified at 17400 rpm for 3 minutes, in 7.5 mL solution 20% w/v of PLGA 752H in ethyl acetate, containing 1.5 g of polymer, by a homogenaizer Ultraturrax t25 basic (IKA), using 8G probe.

The obtained emulsion (W1/O) was quickly added to 30 ml aqueous solution 1% w/v of PVA (Mowiol® 40-88) doped with 900 μL of ethyl acetate and homogenized again at 12000 rpm for 3 minutes. The resulting double emulsion (W1/O/W2) was stirred for 3 hours, at 500 rpm, at room temperature using overhead stirrer RW20D (IKA), with a PTFE-coated-2-bladed propeller stirrer, to allow organic solvent evaporation and PLGA microparticles precipitation. The suspension was collected in two 50 ml PP tubes (falcon) and centrifuged at 7400 rpm, for 15 minutes, at 4° C. The isolated microparticles were washed 6 times using 50 ml milliQ water, by centrifugation at 7000 rpm, 15 minutes and at 4° C. Afterwards, the microparticles were re-suspended in aqueous solution of trehalose 2% w/v. The obtained suspension was moved in ISO 6R glass type siliconised Class 1 vials, and freezed at −80° C. to be, subsequently, lyophilized.

In the same way, a batch of microparticles without rhNGF is prepared (placebo). Instead of rhNGF solution, is used formulation buffer in the phase W1.

Study on the Kinetics of rhNGF Release

The procedure was used to assess the ability of the microparticles to release the rhNGF in the required time (40-60 gg) and to determine the protein released by the mps systems.

### Procedure

Suspend 10 mg of the microparticles in 1 mL of PBS pH 7.4 (Sigma-Aldrich) in 2 mL PP LoBind protein tubes (Eppendorf)
Vortex for 3 sec
Spin the tubes (7500 rpm—7 min—4° C.)
Withdraw totally the vehicle and restore the early volume (1 mL) with fresh medium.
Re-suspend the particles.
Leave the tubes in an incubator shaker at 37° C. under orbital shaking (80 rpm).
Keep the withdrawal samples in the freezer at −20° C., if not immediately analyzed.

It is important to withdraw samples in 3-4 days max, to better preserve the physico-chemical and biological characteristics of rhNGF, which may be compromised because of release kinetics conditions (especially at 37° C.).

### Analysis of Samples by ELISA

The samples should be diluted with Formulation Buffer before being analyzed by ELISA, to ensure that the concentration of NGF is within the limits of detect range of used kit (14-5000 pg/mL). For ELISA analysis is used the kit RayBio® Human beta-NGF, cod. ELH-NGF, provided by Raybiotech, Inc.

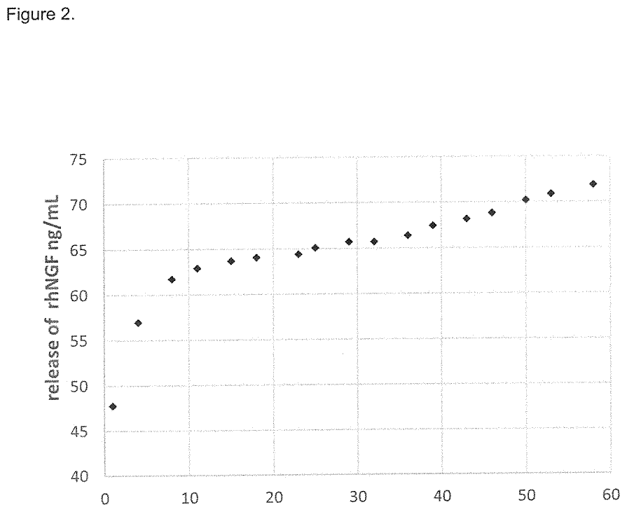

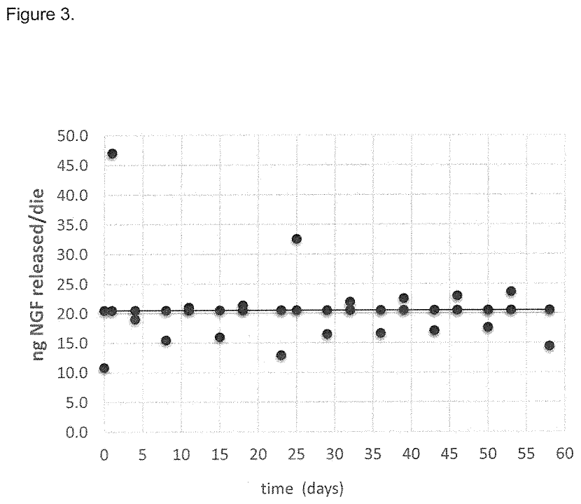

FIGS. 2 and 3 show the rhNGF—MPS in vitro cumulative and day by day (ng/day) release profiles with formulation 2.

### 1. In Vitro Experiment

Cochlear culture represents the standard model to test ability of compound possibly effective in reducing or preventing hearing loss. In the experiment here described, cochlea were extracted from newborn mice (from day 5 to day 15) and damaged with gentamicin treatment (50 μg/ml for 14 hours), Then rhNGF was used at different concentration (6-50-100-300 ng/ml) for 72h. Cell survival was evaluated by Hoechst staining.

Non treated cells (NT), damaged and not treated cells (GNT) and vehicle treated cells were used as controls.

Results

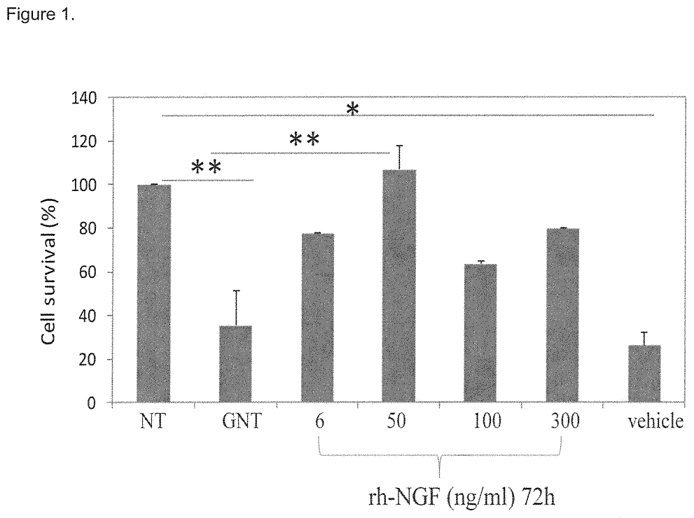

As reported in FIG. 1, the rhNGF treatment of damaged cochlea led to a statistically significant increase in cell survival.

In particular, the data obtained in the experiment demonstrated the rhNGF ability in restoring hair cell viability following damage due to gentamicin exposure in organotypic cultures of the cochlea of mice.

2. Pharmacokinetic of rhNGF after Transtympanic Administration in Guinea Pig

The trans-tympanic injection was carried out on anesthetized animals using a specific endoscope to limit the damage of the tympanic membrane. The drug was administered in the left middle ear of the Guinea Pig, laying on its right side on a heating pad. Perilymph was collected after 0.5, 2, 4, 6 and 24 hours post injection Pharmacokinetic profile of rhNGF after single intravenous (iv) (1.2 mg/kg) and transtympanic (TT) (30 μg/mL) administration was studied in female Albino Hartley guinea pigs.

rhNGF was solubilized in saline buffer and diluted in artificial perilymph to obtain a final concentration.

The trans-tympanic injection was carried out on anesthetized animals using a specific endoscope to limit the damage of the tympanic membrane. The drug was administered in the left middle ear of the Guinea Pig, laying on its right side on a heating pad.

5 μL of cochlear fluid from 5 guinea pigs/time were collected at 0.5, 2, 4, 6 and 24 h after administration. rhNGF was detected in perilymph (ELISA).

In Table 3 the main pharmacokinetic parameters of rhNGF obtained after single i.v. and TT administration are shown.

TABLE 3
Pharmacokinetic parameters of rhNGF after
single i.v. and TT administration

| rhNGF | TT | i.v |
|---|---|---|
| DOSE | 30 μg/mL | 1.2 mg/kg |
| Cmax (ng/mL) | 711.927 | 17.3877 |
| Tmax (h) | 4 | 2 |
| AUClast (μg/mL*min) | 305.595 | 11.4342 |
| T_{1/2} (h) | 17.4846 | 10.1469 |

These data showed that transtympanic injection (TT) is better than i.v. since it results in a Cmax 40-fold and an AUCₜₒₜ (the peak concentration of a drug after administration in perilymph)(Area Under the Curve represents the total drug exposure over time given by summa of AUC last and extra, respectively the AUC of the last dosed time and the AUC of all dosed times after 24 hours from drug exposure) (544,799 μg/mL*min) 38-fold higher with a t_{1/2} roughly 2-fold longer, despite rhNGF dose 40-fold lower in TT than in i.v. administration.

1-16. (canceled)

17. A method of treating hearing disorders in a subject in need thereof, comprising administration of a neurotrophin, wherein the neurotrophin is administered by intratympanic route.

18. The method according to claim 17, wherein the neurotrophin is selected from NGF, brain-derived neurotrophic factor (BDNF), neurotrophin 3 and neurotrophin 4.

19. The method according to claim 18, wherein the neurotrophin is NGF.

20. The method according to claim 17, wherein the hearing disorder is selected from sudden deafness, blast-induced hearing loss, ototoxicity, ARHL (age related hearing loss) and noise damage.

21. The method according to claim 17, wherein the neurotrophin is administered in the form of a composition comprising the neurotrophin and at least a pharmaceutically acceptable excipient.

22. The method according to claim 21, wherein the pharmaceutically acceptable excipient is selected from hyaluronic acid, polyvinyl alcohol, glycerol, poly-lactic-co-glycolic acid and PEG400.

23. The method according to claim 22, wherein the pharmaceutically acceptable excipient is hyaluronic acid, polyvinyl alcohol or glycerol.

24. The method according to claim 21, wherein the composition is a solution, a suspension, microparticles or a gel.

25. The method according to claim 24, wherein the microparticles are of PLGA.

26. The method according to claim 17, wherein the neurotrophin is administered simultaneously, separately or sequentially in combination with a further active principle.

27. The method according to claim 26, wherein the further active principle is a steroid selected from dexamethasone, methylprednisolone and prednisolone.

28. The method according to claim 21, wherein the composition contains from 1 μg to 1.2 μg of NGF.

29. The method according to claim 28, wherein the composition contains from 10 μg to 200 μg of NGF.

30. The method according to claim 29, wherein the composition contains from 2 μg to 20 μg of NGF.

31. The method according to claim 17, wherein the neurotrophin is of murine or human origin or it is a human recombinant neurotrophin.

32. The method according to claim 31, wherein the neurotrophin is a NGF of murine or human origin or it is a human recombinant NGF.

33. The method according to claim 17, wherein the neurotrophin is administered daily.

34. The method according to claim 33, wherein the neurotrophin is administered one or more times a day.
